(19) United States
(12) Patent Application Publication
(10) Pub. No.: US 2006/0009672 A1
(43) Pub. Date: Jan. 12, 2006

(54) Atrial fibrillation treatment and method

(76) Inventor: Sumit Verma, Pensacola, FL (US)

Correspondence Address: S. Michael Bender, P.O. Box 530-399, St. Petersburg, FL, 33747, US

(21) Appl. No.: 11/179,153

(22) Filed: Jul. 12, 2005

Related U.S. Application Data
(60) Provisional application No. 60/586,505, filed on Jul. 12, 2004.

(51) Int. Cl. A61N 1/362; A61M 1/10; A61H 19/00

(52) U.S. Cl. 600/16; 623/3.11; 601/153

(57) ABSTRACT

An apparatus is provided for compressing and permitting relaxation of a body organ in a body. In general, the apparatus includes organ compression means attached to a portion of the body organ for compressing the body organ, and compression activation means are connected to the organ compression means for controlling the organ compression means for compressing the body organ and for permitting the compressed body organ to relax. With one class of embodiments of the invention, the organ compression means include electromagnet means, and the compression activation means are connected to the electromagnet means and include an electromagnet control unit for controlling the electromagnet means. With another class of embodiments of the invention, the organ compression means include a pneumatic bladder, and the compression activation means are connected to the pneumatic bladder, includes a pneumatic bladder control unit for controlling the pneumatic bladder. A method is also provided for compressing and permitting relaxation of a body organ.

### CROSS-REFERENCE TO RELATED APPLICATION

This application claims priority based upon my copending Provisional Application Ser. No. 60/586,505, filed Jul. 12, 2004, entitled: Atrial Assist Device for Treatment of Chronic Fibrillation, which provisional application hereby is incorporated herein by this reference.

### BACKGROUND OF THE INVENTION

1. Field of the Invention

The present invention relates generally to methods and devices for treating cardiac arrhythmias, and, more particularly, to a method and apparatus especially adapted for treating left atrial fibrillation.

2. Description of the Prior Art

The path of blood flow in and out of the human heart is well known. Briefly, deoxygenated blood flows from the body into the right atrium, then into the right ventricle, then to the lungs where the blood is oxygenated, then oxygenated blood flows from the lungs into the left atrium, then into the left ventricle, and then throughout the body.

More specifically, it is well known that loss of left atrial contraction due to left atrial fibrillation can result in symptoms of fatigue, dyspnea, and congestive heart failure. Current therapies aimed at treatment of left atrial fibrillation are only partially successful, and curative treatment is elusive.

Left atrial fibrillation affects nearly 1 in 10 people over 80 years of age. Atrial fibrillation is a leading cause of congestive heart failure, stroke, and mortality, especially in the aging population. It is one of the most common problems in elderly patients. In patients who have long standing atrial fibrillation, frequently atrial fibrillation has to be accepted as there is no treatment available when the atrium is severely dilated and diseased. Atrial fibrillation causes more than 70,000 strokes each year in the U.S., where 160,000 new cases of Atrial Fibrillation are diagnosed each year. Atrial fibrillation accounts for one fourth of all strokes in the elderly. Atrial fibrillation affects more than 2.2 million people in the U.S., and this number will double within the next 20 years. Atrial fibrillation can also decrease the cardiac output by as much as 20 to 30 percent. This percentage of decrease in cardiac output is higher in patients with abnormal left ventricular function where atrial contraction can contribute to a decrease of 50% of the cardiac output.

With respect to therapies for left atrial fibrillation, antiarrhythmic therapies have possible proarrhythmic effects and other side effects that are potentially life threatening such as pulmonary toxicity and amiodarone, Torsades de pointes with Sotalol, etc.

Surgical options such as the MAZE procedure have a higher degree of success, but require a thoracotomy and cardiopulmonary bypass.

The same is true for catheter ablation. Newer catheter ablation approaches for left atrial fibrillation (such as circumferential left atrial catheter ablation described by Dr. Pappone) shoe promise, but they are difficult to perform and will likely never be widely available. In addition, complications such as Esophageo-atrial fistula occurring with this approach are catastrophic. Also, catheter ablation is frequently required more than once (50% of patients require a second ablation) to achieve control of left atrial fibrillation.

Even in the most experienced hands, the success rates of catheter ablation are about 70% only for patients with Paroxysmal atrial fibrillation (not atrial fibrillation associated with other cardiac disease). A large percentage of patients with atrial fibrillation have an associated cardiomyopathy. Recently, it has been shown that patients with congestive heart failure and ejection fraction of less than 45% do better with ablation. However, the ablation approach is complicated and needs to be redone in several patients. Hence, although ablation approach is attractive, it is complicated, risky, and there are very few operators who can do this procedure. Certainly, there are not enough operators to deal with the large number of patients with atrial fibrillation.

Patients with dilated left atria and long standing left atrial fibrillation have a low chance of restoring sinus rhythm and chronic atrial fibrillation has to be accepted.

Frequently, despite aggressive treatment, patients do not regain sinus rhythm. Moreover, even if electrical sinus rhythm is regained, the patients do not regain mechanical contraction of the left atrium. The patients must still take anticoagulants (coumadin) to prevent a stroke and remain symptomatic. If these patients have other heart disease such as Left ventricular hypertrophy, hypertrophic cardiomyopathy, aortic stenosis, or left ventricular systolic dysfunction, the persistent loss of left atrial function may result in recurrent congestive heart failure and death.

For the categories of patients described hereinabove, there is currently no available therapy which targets the loss of left atrial function. In this respect, a therapy is needed that targets the loss of left atrial function. More specifically, what is needed is a simple, predictable, and effective non-pharmacological therapy that targets left atrial function.

Still other features would be desirable in an atrial fibrillation treatment method and apparatus. For example, it would be desirable to have an apparatus that is simple and easy to implant and will restore contractility of the left atrium in patients who have chronic atrial fibrillation (or sinus rhythm but poor atrial contraction due to atrial myopathy) that is refractory to medical or ablative therapy. Also, it would be desirable to have an apparatus that can be implanted epicardially at the time of cardiac surgery. Furthermore, it would be desirable to have an apparatus that is positioned outside the left atrium and will not itself be a source of thromboembolism, and may well result in a decrease in the risk of thromboembolism from atrial fibrillation. Also, it would be desirable to have an apparatus that allows programmable timing of the contraction of the left atrium to allow optimal left ventricular filling. In addition, it would be desirable to have a method that allows left atrial contraction using a method of electromagnetic coupling. Further, it would be desirable to have an apparatus that allows incorporation into currently available biventricular pacemakers and defibrillators.

Thus, while the foregoing body of prior art indicates it to be well known to use methods to reduce atrial fibrillation, the prior art described above does not teach or suggest an atrial fibrillation treatment method and apparatus which has the following combination of desirable features: (1) targets the loss of left atrial function; (2) is a simple, predictable, and effective non-pharmacological therapy that targets left atrial function; (3) is simple and easy to implant and will restore contractility of the left atrium in patients who have chronic atrial fibrillation (or sinus rhythm but poor atrial contraction due to atrial myopathy) that is refractory to medical or ablative therapy; (4) can be implanted epicardially at the time of cardiac surgery; (5) is positioned outside the left atrium and will not itself be a source of thromboembolism, and may well result in a decrease in the risk of thromboembolism from atrial fibrillation; (6) allows programmable timing of the contraction of the left atrium to allow optimal left ventricular filling; (7) allows left atrial contraction using a method of electromagnetic coupling; and (8) allows incorporation into currently available biventricular pacemakers and defibrillators. The foregoing desired characteristics are provided by the unique atrial fibrillation treatment method and apparatus of the present invention as will be made apparent from the following description thereof. Other advantages of the present invention over the prior art also will be rendered evident.

### SUMMARY OF THE INVENTION

To achieve the foregoing and other advantages, the present invention, briefly described, provides an apparatus for compressing and permitting relaxation of a body organ in a body. In general, the apparatus includes organ compression means attached to a portion of the body organ for compressing the body organ, and compression activation means are connected to the organ compression means for controlling the organ compression means for compressing the body organ and for permitting the compressed body organ to relax.

With one class of embodiments of the invention, the organ compression means include electromagnet means, and the compression activation means are connected to the electromagnet means and include an electromagnet control unit for controlling the electromagnet means.

With a first embodiment of the invention, the electromagnet means include a first electromagnet that is electrically connected to the electromagnet control unit. The first electromagnet is placed on a first organ location on the body organ, and a second electromagnet is electrically connected to the electromagnet control unit. The second electromagnet is placed on a second organ location on the body organ. The first electromagnet and the second electromagnet are independently controllable by the electromagnet control unit, such that the first electromagnet and the second electromagnet control compression of and permit relaxation of the body organ.

The body organ can be a heart; the first organ location can be the left atrial surface of the heart; the second organ location can be the parietal pericardium of the heart, and the first electromagnet and the second electromagnet are independently controlled by the electromagnet control unit, such that they control a left atrial contraction and permit relaxation of the left atrium.

With a second embodiment of the invention, the electromagnet means include an outer first electromagnet supported by a relatively stiff, nondeformable outer first-electromagnet-support member. An inner second electromagnet is supported by a flexible and deformable inner second-electromagnet-support member. Preferably, the outer first-electromagnet-support member and the inner second-electromagnet-support member are formed as a unified, integrated structure, and the unified integrated structure is formed as a patch that is attached to the body organ. A quantity of inert gas can be contained inside the patch between the outer first-electromagnet-support member and the inner second-electromagnet-support member. The patch can be sutured to the left atrial surface or the left ventricular surface, such that repulsion of the second electromagnet by the first electromagnet causes an inward compression of the left atrium.

With a third embodiment of the invention, the electromagnet means include a flexible base member, and a plurality of electromagnet rings attached concentrically to the flexible base member.

With a fourth embodiment of the invention, the electromagnet means include a flexible linear first electromagnet placed on a first organ location, and a patch-like second electromagnet is placed on a second organ location. The flexible linear first electromagnet can be placed on the epicardial surface of the left atrium, and the patch-like second electromagnet can be placed in either the coronary sinus or the intraatrial septum.

With another class of embodiments of the invention, and with a fifth embodiment of the invention, the organ compression means include a pneumatic bladder, and the compression activation means are connected to the pneumatic bladder, includes a pneumatic bladder control unit for controlling the pneumatic bladder.

With respect to another aspect of the invention, a method is provided for compressing and permitting relaxation of a body organ includes the steps of:
attaching a portion of organ compression means to the body organ, and
employing compression activation means for controlling the organ compression means for compressing and permitting relaxation of the body organ.

More specifically, the method of the invention can include the steps of:
placing a first electromagnet, which is part of the organ compression means, on a first organ location of the body organ, placing a second electromagnet, which is part of the organ compression means, on a second organ location of the body organ, and
independently controlling the first electromagnet and the second electromagnet by an electromagnet control unit, which is part of the compression activation means, such that the first electromagnet and the second electromagnet compress and permit relaxation of a portion of the body organ.

Further, with the method of the invention, the body organ is a heart; the first organ location is the left atrial surface of the heart; the second organ location is the parietal pericardium of the heart; and the first electromagnet and the second electromagnet are independently controlled by the electromagnet control unit such that they control a left atrial contraction and permit relaxation of the left atrium.

The above brief description sets forth rather broadly the more important features of the present invention in order that the detailed description thereof that follows may be better understood, and in order that the present contributions to the art may be better appreciated. There are, of course, additional features of the invention that will be described hereinafter and which will be for the subject matter of the claims appended hereto.

In this respect, before explaining at least five preferred embodiments of the invention in detail, it is understood that the invention is not limited in its application to the details of the construction and to the arrangements of the components set forth in the following description or illustrated in the drawings. The invention is capable of other embodiments and of being practiced and carried out in various ways. Also, it is to be understood, that the phraseology and terminology employed herein are for the purpose of description and should not be regarded as limiting.

As such, those skilled in the art will appreciate that the conception, upon which disclosure is based, may readily be utilized as a basis for designing other structures, methods, and systems for carrying out the several purposes of the present invention. It is important, therefore, that the claims be regarded as including such equivalent constructions insofar as they do not depart from the spirit and scope of the present invention.

It is therefore an object of the present invention to provide a new and improved atrial fibrillation treatment method and apparatus which has all of the advantages of the prior art and none of the disadvantages.

It is another object of the present invention to provide a new and improved atrial fibrillation treatment method and apparatus which may be easily and efficiently manufactured and marketed.

It is a further object of the present invention to provide a new and improved atrial fibrillation treatment method and apparatus which is of durable and reliable construction.

An even further object of the present invention is to provide a new and improved atrial fibrillation treatment method and apparatus which is susceptible of a low cost of manufacture with regard to both materials and labor, and which accordingly is then susceptible of low prices of sale to the consuming public, thereby making such atrial fibrillation treatment method and apparatus available to the buying public.

Still yet a further object of the present invention is to provide a new and improved atrial fibrillation treatment method and apparatus which targets the loss of left atrial function.

Still another object of the present invention is to provide a new and improved atrial fibrillation treatment method and apparatus that is a simple, predictable, and effective non-pharmacological therapy that targets left atrial function.

Still a further object of the present invention is to provide a new and improved atrial fibrillation treatment method and apparatus that is simple and easy to implant and will restore contractility of the left atrium in patients who have chronic atrial fibrillation (or sinus rhythm but poor atrial contraction due to atrial myopathy) that is refractory to medical or ablative therapy.

Yet another object of the present invention is to provide a new and improved atrial fibrillation treatment method and apparatus that can be implanted epicardially at the time of cardiac surgery.

Still a further object of the present invention is to provide a new and improved atrial fibrillation treatment method and apparatus that is positioned outside the left atrium and will not itself be a source of thromboembolism, and may well result in a decrease in the risk of thromboembolism from atrial fibrillation. This feature may allow discontinuation of anticoagulation therapy after efficacy is confirmed in clinical tests.

Still another object of the present invention is to provide a new and improved atrial fibrillation treatment method and apparatus that allows programmable timing of the contraction of the left atrium to allow optimal left ventricular filling. The atrial electrode of the subject invention allows sensing and pacing of the atrium to allow contraction therapy during atrial fibrillation only. If the patient achieves sinus rhythm, the subject apparatus will function as a pacing electrode.

Yet another object of the present invention is to provide a new and improved atrial fibrillation treatment method and apparatus that allows left atrial contraction using a method of electromagnetic coupling. This allows a reasonable sized implantable battery source to be used. A similar effect can be achieved with pneumatically controlled contraction.

Still a further object of the present invention is to provide a new and improved atrial fibrillation treatment method and apparatus that allows incorporation into currently available biventricular pacemakers and defibrillators.

These together with still other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE DRAWINGS

The invention will be better understood and the above objects as well as objects other than those set forth above will become more apparent after a study of the following detailed description thereof. Such description makes reference to the annexed drawing wherein:

FIG. 1 shows a first embodiment of the invention in perspective wherein a first electromagnet is placed on the left atrial surface, and a second electromagnet, physically separated from the first electromagnet, is sutured to the parietal pericardium of the heart.

FIG. 2 is an enlarged side view of the embodiment of the invention shown in FIG. 1 illustrating how the first and second electromagnets are physically separated from each other.

FIG. 3 shows a second embodiment of the invention wherein an outer first-electromagnet-support member and an inner second-electromagnet-support member are formed as a unified, integrated structure which is attached to the heart in the form of a patch.

FIG. 4 is an enlarged side view of the embodiment of the invention shown in FIG. 3 removed from the heart.

FIG. 5 shows a third embodiment of the invention wherein a plurality of electromagnet rings are attached concentrically to a flexible base member, which is attached to the heart in the form of another patch.

FIG. 6 is an enlarged side view of the embodiment of the invention shown in FIG. 5 removed from the heart.

FIG. 7 shows a fourth embodiment of the invention wherein a flexible linear first electromagnet is placed on a first heart location, and a patch-like second electromagnet is placed on a second heart location.

FIG. 8 is an enlarged, exploded side view of the embodiment of the invention shown in FIG. 7 removed from the heart.

FIG. 9 shows a fifth embodiment of the invention wherein a pneumatic bladder is attached to the left atrium and a pneumatic bladder control unit controls the pneumatic bladder.

FIG. 10 is an enlarged side view of the embodiment of the invention shown in FIG. 9 removed from the heart.

FIG. 11 shows a sixth embodiment of the invention wherein a first electromagnet is placed on the inside atrial surface via venous transplantation, and a second electromagnet, physically separated from the first electromagnet, is placed on the outside of the parietal pericardium of the heart.

FIG. 12 is an enlarged side view of the embodiment of the invention shown in FIG. 11 illustrating how the first and second electromagnets are physically separated from each other.

FIG. 13 shows a seventh embodiment of the invention suitable for treating the ventricular wall portion a heart wherein a series of first electromagnets are placed on the ventricular wall surface, and a second series of electromagnets, physically separated from the first electromagnet, can suitably be associated therewith to treat a diseased or damaged portion of the left ventricle of the heart.

FIG. 14 is an enlarged side view of the embodiment of the invention shown in FIG. 13 illustrating how the first and second series of electromagnets are physically oriented with respect to each other and the left ventricle of a heart.

### DESCRIPTION OF THE PREFERRED EMBODIMENT

With reference to the drawings, new and improved atrial fibrillation treatment methods and apparatuses embodying the principles and concepts of the present invention will be described.

In general, an apparatus is provided for compressing and permitting relaxation of a body organ in a body. The apparatus includes organ compression means attached to a portion of the body organ for compressing the body organ, and compression activation means are connected to the organ compression means for controlling the organ compression means for compressing the body organ and for permitting the compressed body organ to relax.

With a first class of embodiments of the invention, the organ compression means include electromagnet means, and the compression activation means are connected to the electromagnet means and include an electromagnet control unit for controlling the electromagnet means.

With all drawing figures described below, reference numerals are shown that correspond to like reference numerals that designate like elements shown in other drawing figures.

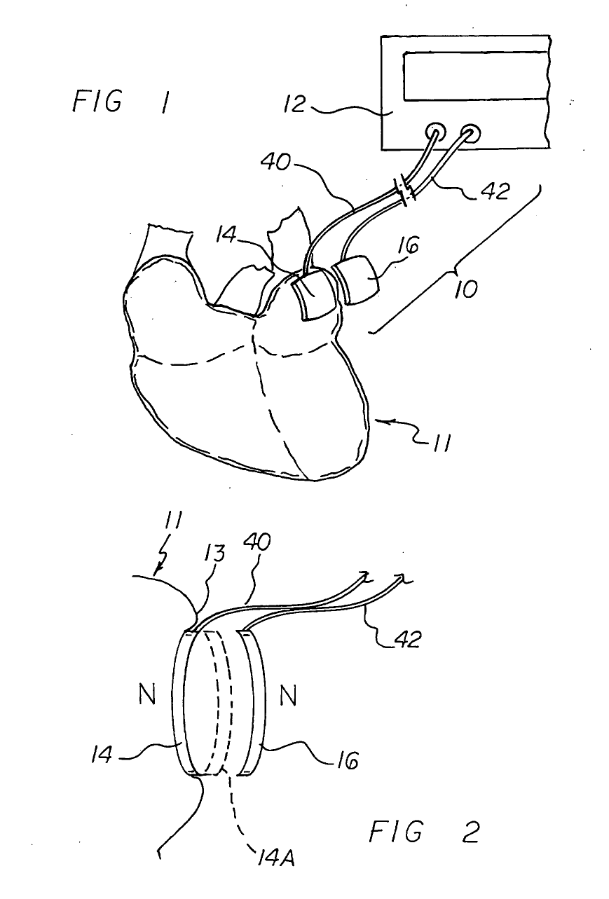

With one embodiment of the invention 10, referring to FIGS. 1 and 2, the electromagnet means include a first electromagnet 14 that is electrically connected through a first insulated conductor 40 to the electromagnet control unit 12. The first electromagnet 14 is placed on a first organ location on the body organ, and a second electromagnet 16 is electrically connected to the electromagnet control unit 12. The second electromagnet 16 is placed on a second organ location on the body organ. The first electromagnet 14 and the second electromagnet 16 are independently controllable by the electromagnet control unit 12, such that the first electromagnet 14 and the second electromagnet 16 control compression of and permit relaxation of the body organ.

The body organ can be a heart 11; the first organ location can be the left atrial surface of the heart; the second organ location can be the parietal pericardium of the heart, and the first electromagnet 14 and the second electromagnet 16 are independently controlled by the electromagnet control unit 12, such that they control a left atrial contraction and permit relaxation of the left atrium. The electromagnet control unit 12 can be an adapted pacemaker.

More specifically, with this embodiment, the first electromagnet 14 is placed on the left atrial surface 13, and the second electromagnet 16 sutured to the parietal pericardium of the heart. Referring to FIG. 2, the activated state of the first electromagnet 14 is shown in solid lines causing the left atrium to be compressed. The deactivated state of the first electromagnet 14 is shown in broken lines 14a, wherein the left atrium can return to a relaxed condition.

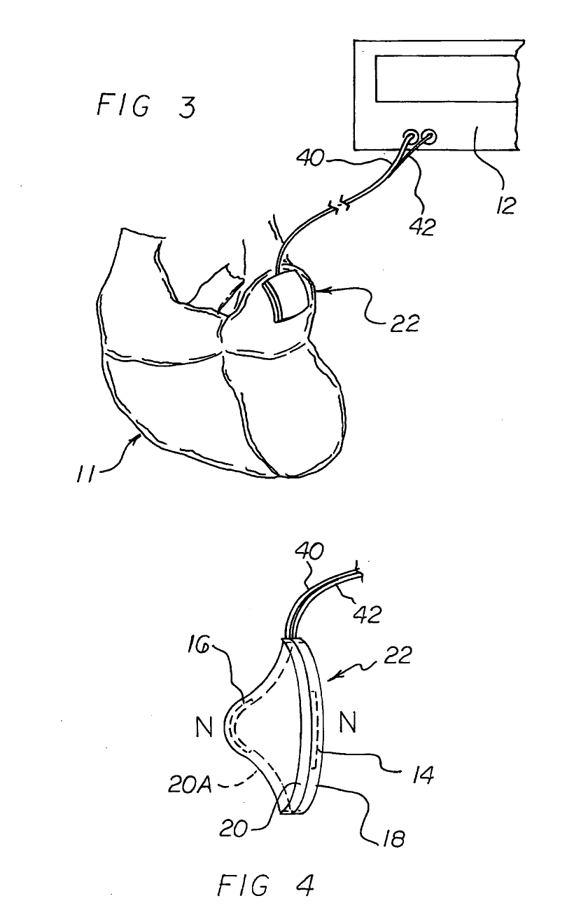

With another embodiment of the invention, referring to FIGS. 3 and 4, the electromagnet means include an outer first electromagnet 14 supported by a relatively stiff, nondeformable outer first-electromagnet-support member 18. An inner second electromagnet 16 is supported by a flexible and deformable inner second-electromagnet-support member 20.

Preferably, the outer first-electromagnet-support member 18 and the inner second-electromagnet-support member 20 are formed as a unified, integrated structure, and the unified integrated structure is formed as a patch 22 that is attached to the body organ.

A quantity of inert gas can be contained inside the patch 22 between the outer first-electromagnet-support member 18 and the inner second-electromagnet-support member 20. The state of magnetic repulsion between the first electromagnet 14 and the second electromagnet 16 is shown in FIG. 4 wherein the inner second-electromagnet-support member 20 is shown in broken lines 20a to be displaced from the outer first-electromagnet-support member 18. In contrast, when the second electromagnet 16 is either inactivated or in a magnetic attractive state with respect to the first electromagnet 14, the inner second-electromagnet-support member 20 returns to a location adjacent to the outer first-electromagnet-support member 18, wherein the inner second-electromagnet-support member 20 is shown in solid lines 20.

The patch 22 can be sutured to the left atrial surface or the left ventricular surface, such that repulsion of the second electromagnet 16 by the first electromagnet 14 causes an inward compression of the left atrium. The degree of displacement of volume in the left atrium is controllable by controlling the electromagnetic repulsion between the second electromagnet 16 and the first electromagnet 14. In addition, a pressure sensor (not shown) and a temperature sensor (not shown) can be attached to the inner second-electromagnet-support member 20 to allow automatic inactivation/activation of the repulsion/attraction between the second electromagnet 16 and the first electromagnet 14 when predetermined pressure and temperature parameters have been attained.

Once the electromagnetic repulsion between the second electromagnet 16 and the first electromagnet 14 is ceased, the left atrium can relax and return to normal. If desired, once the electromagnetic repulsion is ceased, an electromagnetic attraction can be carried out to facilitate the return of the left atrium to the precompression state.

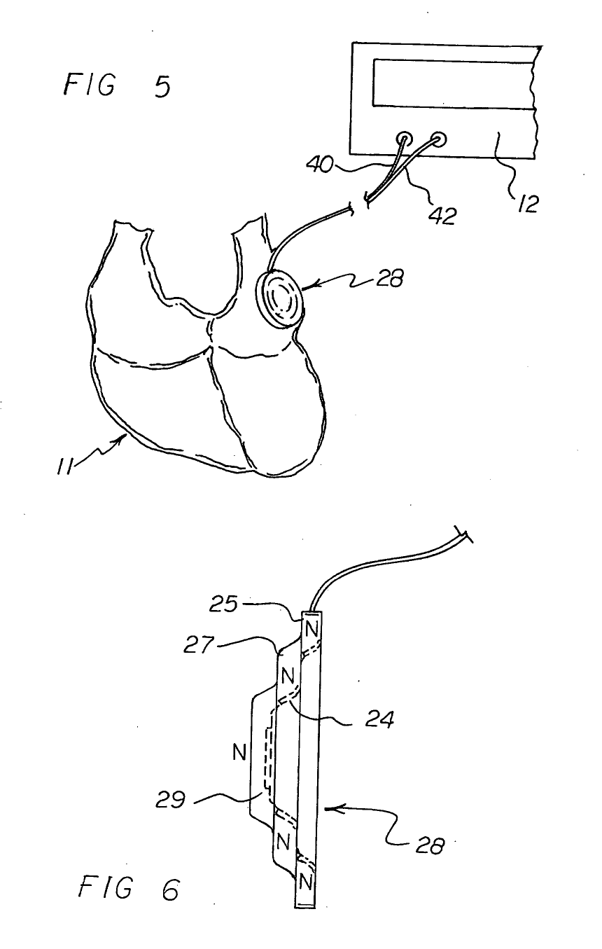

With another embodiment of the invention, referring to FIGS. 5 and 6, the electromagnet means include a flexible base member 24, and a plurality of electromagnet rings 25, 27, 29 attached concentrically to the flexible base member 24. The flexible base member 24 and the electromagnet rings 25, 27, 29 can be provided as another patch 28. This other patch 28 can be sutured to the heart, and the electromagnet rings 25, 27, 29 are controlled by the electromagnet control unit 12 so that the left atrium is caused to be compressed and then allowed to relax to overcome atrial fibrillation. If desired, instead of using a flexible base member 24 for securing the electromagnet rings 25, 27, 29 to one another, each individual electromagnet ring 25, 27, 29 can be attached to respective adjacent rings by flexible ring-to-ring attachment means.

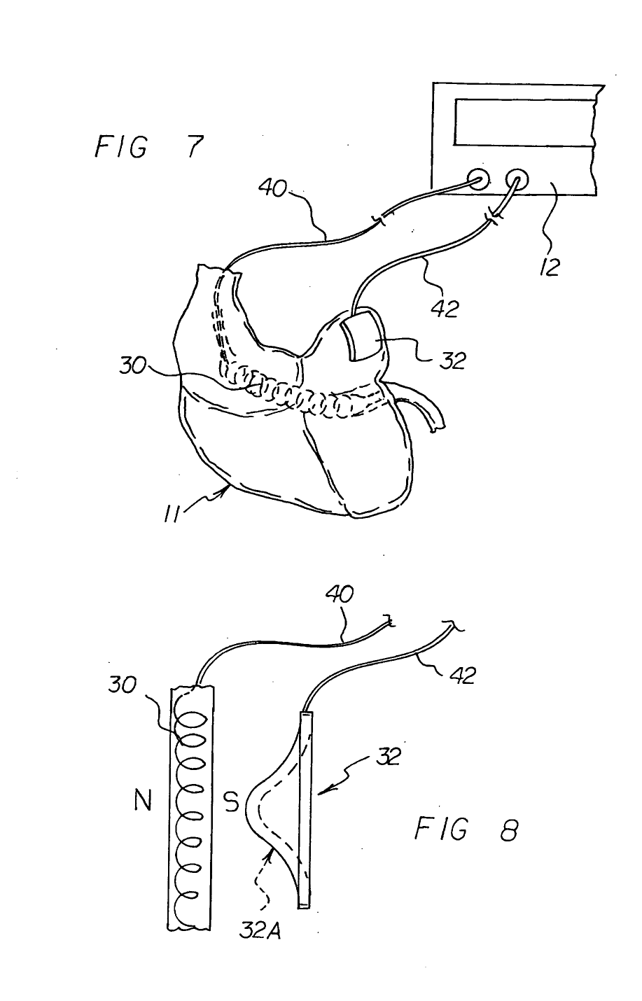

With another embodiment of the invention, referring to FIGS. 7 and 8, the electromagnet means include a flexible linear first electromagnet 30 placed on a first organ location, and a patch-like second electromagnet 32 is placed on a second organ location.

The flexible linear first electromagnet 30 can be placed on the epicardial surface of the left atrium, and the patch-like second electromagnet 32 can be placed in either the coronary sinus or the intraatrial septum. The patch-like second electromagnet 32 can be placed in the intraatrial septum with a procedure which is similar to placement of an atrial septal defect closure device. This patch-like second electromagnet 32 is reversibly magnetized by passage of current into the patch-like second electromagnet 32 through a electromagnet control unit 12, which can be similar to a pacemaker system. The timing of this current is such that optimal filling of the left ventricle can result.

In FIG. 8, the relaxed state of the patch-like second electromagnet 32 is shown in solid lines 32. In this state either the patch-like second electromagnet 32 is not attracted to the flexible linear first electromagnet 30, or the patch-like second electromagnet 32 is repelled from the flexible linear first electromagnet 30. With the patch-like second electromagnet 32 in the deactivated state, the left atrium of the heart 11 is not compressed. The activated state of the patch-like second electromagnet 32 is shown in broken lines 32a, wherein the patch-like second electromagnet 32 is attracted to the flexible linear first electromagnet 30, and whereby the left atrium of the heart 11 is compressed.

In addition, a number of procedural steps are followed for placement of the patch-like second electromagnet 32 in the coronary sinus as described below. The patch-like second electromagnet 32 is placed in the coronary sinus using a transvenous approach such as used with a left ventricular pacing lead placement. The patch-like second electromagnet 32 is fit into a 7-8 Fr catheter system or even smaller if possible. The electromagnet coils can be rotated inside the catheter to allow best alignment. The electromagnet coils of the patch-like second electromagnet 32 create magnetic field to attract the flexible linear first electromagnet 30 that is placed on the epicardial surface of the left atrium.

Optionally, but not preferably, either the flexible linear first electromagnet 30 or the patch-like second electromagnet 32 can be substituted with a permanent magnet rather than an electromagnet. However, with a permanent magnet, if the patient is exposed to an environmental magnetic field, then such an environmental magnetic field may undesirably interact with the permanent magnet, causing undesirable effects on the patient.

When both the flexible linear first electromagnet 30 and the patch-like second electromagnet 32 are employed, the respective magnetic fields can be controlled and reversed so that environmental magnetic fields do not exert undesirable effects.

A system that promotes free blood flow around the catheter, possibly a catheter with surface undulations, will prevent overheating of the catheter due to cycling magnetic fields. More specifically, the coronary sinus is a high flow vein, and it has been noted that during radiofrequency ablation with the coronary sinus, it can be difficult to achieve high temperatures. This cooling characteristic of the coronary sinus with allow for spontaneous cooling of the catheter.

The catheter has an internal lumen to permit over the wire placement. This lumen may or may not be large enough to permit a 5 Fr pacing wire. The catheter should have a method to stabilize it with the coronary sinus so that there is no significant movement of the catheter. To assure lack of movement of the catheter. A fixation device, such as an inflatable or expandable balloon, can be employed.

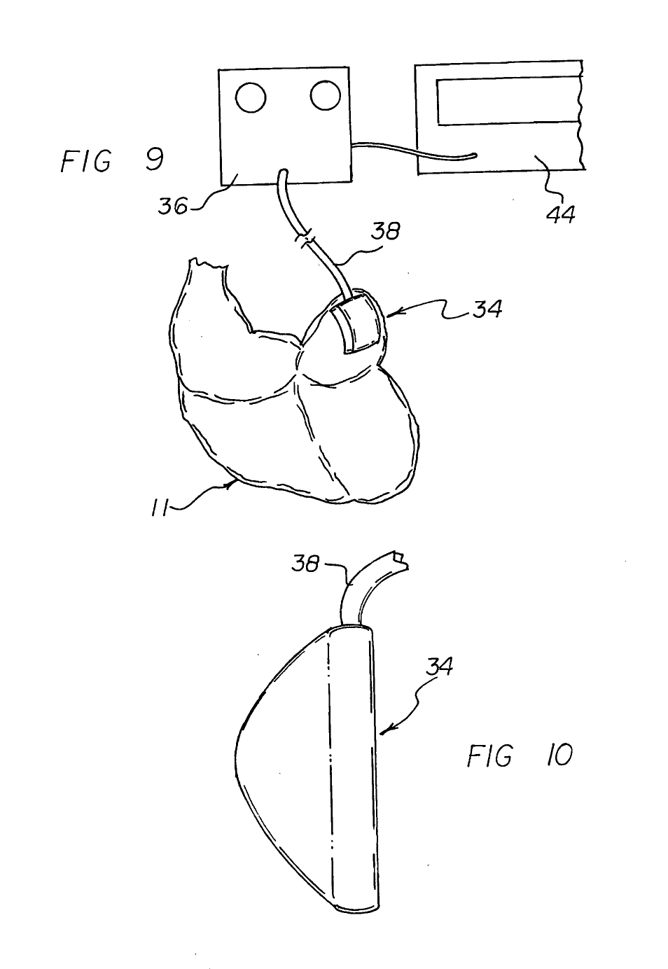

With a second class of embodiments of the invention, as shown in FIGS. 9 and 10, the organ compression means include a pneumatic bladder 34, and the compression activation means are connected to the pneumatic bladder 34. The compression activation means include a pneumatic bladder control unit 36 for controlling the inflation/deflation of the pneumatic bladder 34. The pneumatic bladder control unit 36 is controlled by an electrical control unit 44.

The pneumatic bladder 34 can be in the form of a patch that is sutured onto the external surface of the left atrium during a thoracotomy. Alternatively, the pneumatic bladder 34 is otherwise secured to the left atrium or to the surrounding cardiac structures. The outer surface of the pneumatic bladder 34 faces the pericardium, and the inner surface of the pneumatic bladder 34 faces the left atrium. Inflation and deflation of the pneumatic bladder 34 is controlled by pressurized gas, e.g. air, that flows from the pneumatic bladder control unit 36 through tubing 38 to the pneumatic bladder 34. Preferably, the pneumatic bladder control unit 36 is implanted in the chest. Inflation of the pneumatic bladder 34 causes expansion thereof, and the expansion of the pneumatic bladder 34 causes compression of the left atrium. Deflation of the pneumatic bladder 34 permits relaxation and filling of the left atrium.

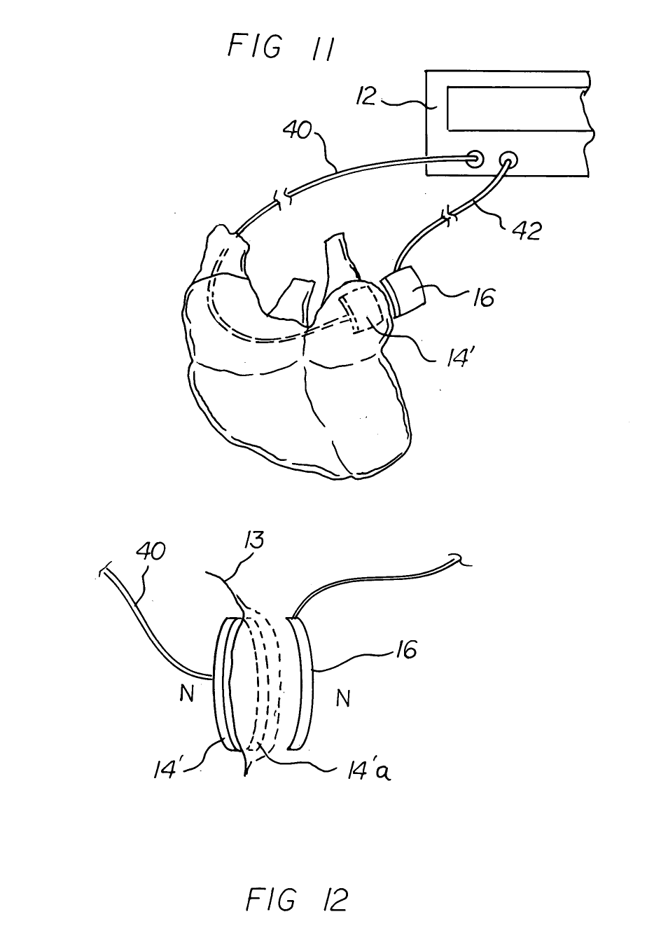

Still other variations within the spirit of the invention are contemplated. Thus, with yet another embodiment of the invention illustrated in FIGS. 11 and 12, the first electromagnet 14′ may be placed on the inside surface of the left atrial surface 13, and the second electromagnet 16 is placed proximal thereto and the parietal pericardium of the heart. Referring to FIG. 12, the de-activated state of the first electromagnet 14′ is shown in solid lines causing the left atrium to be relaxed. The activated state of the first electromagnet 14′ is shown in broken lines 14′a, wherein the left atrium can placed in a compressed condition. The advantage of this alternative arrangement (FIGS. 11 and 12) is that the atrial coil or electromagnetic may be implanted transvenously via transeptal puncture thus allowing placement of the electromagnet without the necessity of an open chest surgical procedure.

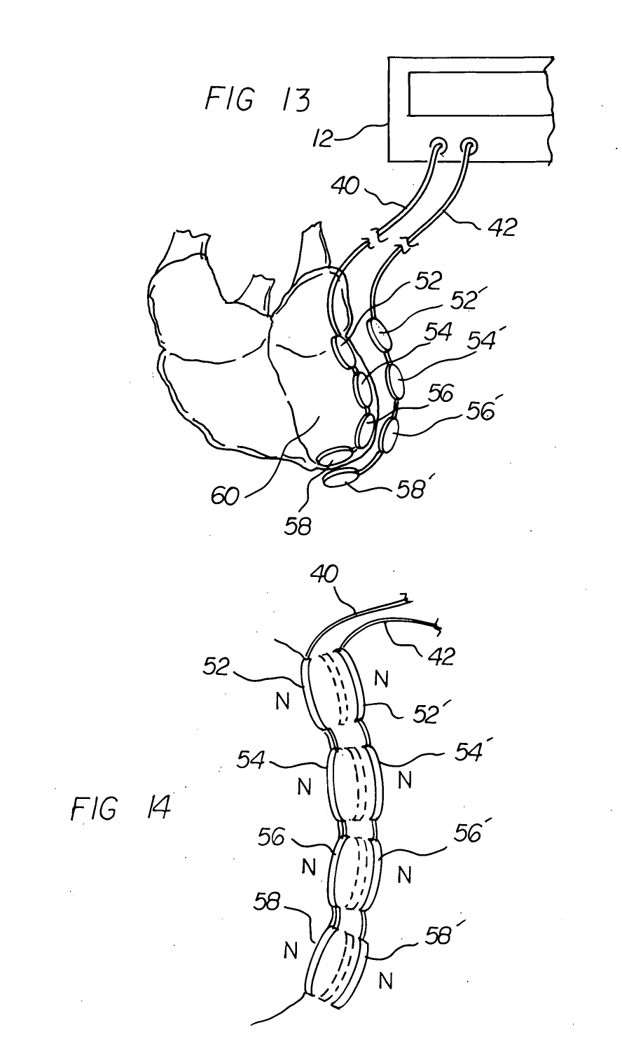

Similarly, as illustrated in FIGS. 13 and 14, a string of serially connected first electromagnets 52, 54, 56, and 58 can be disposed suitably on the left ventricular surface 60 of a heart and an accompanying or second series of corresponding electromagnets 52′, 54′, 56′ 58′ can be disposed proximally substantially as depicted. By means of the electromagnet control unit 12, selected pairs of electromagnets 52, 52′; 54, 54′; 56, 56′; and 58, 58′ may be controlled suitably to provide compression of and relaxation of a body organ such as the left ventricle of a heart. Hence, it will be appreciated that by this alternative arrangement (FIGS. 13 and 14), there is provided an effective means for providing regional support (i.e. assist contraction) of a diseased or damaged wall of the left ventricle only, by selective control of a series of electromagnets.

With respect to another aspect of the invention, a method is provided for compressing and permitting relaxation of a body organ includes the steps of:
attaching a portion of organ compression means to the body organ, and
employing compression activation means for controlling the organ compression means for compressing and permitting relaxation of the body organ.

More specifically, the method of the invention can include the steps of:
placing a first electromagnet 14, which is part of the organ compression means, on a first organ location of the body organ, placing a second electromagnet 16, which is part of the organ compression means, on a second organ location of the body organ, and independently controlling the first electromagnet 14 and the second electromagnet 16 by an electromagnet control unit 12, which is part of the compression activation means, such that the first electromagnet 14 and the second electromagnet 16 compress and permit relaxation of a portion of the body organ.

Further, with the method of the invention, the body organ is a heart; the first organ location is the left atrial surface of the heart; the second organ location is the parietal pericardium of the heart; and the first electromagnet 14 and the second electromagnet 16 are independently controlled by the electromagnet control unit 12 such that they control a left atrial contraction and permit relaxation of the left atrium.

If desired the electromagnet apparatus of the invention can be adapted for cardiac chambers besides the atrium, and the invention can even be adapted for other organs, for example, gall bladder, urinary bladder, etc.

It is anticipated that the atrial fibrillation treatment method and apparatus of the invention interact with the physiology of the left atrium in a number of ways as described below. Citation numbers, that are bracketed in parentheses herein, refer to the list of references provided below.

Left atrial function has received considerably less attention than has left ventricular function even though evidence suggest that left atrial myopathy and failure may exist in an isolated entity, precede left ventricular myopathy, or coexist with left ventricular myopathy and failure (1).

Impaired exercise tolerance is one of the most common clinical manifestations in patients with left ventricular dysfunction (5). Left atrial fractional shortening at res reflects left ventricular filling during exercise and therefore predicts cardiac output and stroke volume response to exercise, and exercise capacity (6). Exercise and left ventricular performance during exercise have been shown to be mainly dependent on left ventricular diastolic filling rather than systolic function (6). Of note, it has been reported that indexes of left atrial function are related to peak aerobic capacity in patients with congestive heart failure (7).

The left atrium is roughly 4 corn in the anteroposterior axis, however in diseased states may enlarge to up to 7 cm. The chamber receives blood from the pulmonary veins and transmits it to the left ventricle. The left atrium is described to have a reservoir function, conduit function and a transport function (2).

Atrial fibrillation and atrial myopathies cause loss of left atrial transport function and due to this blood flow is passive and forces elevation of the left atrial pressure. This may result in pulmonary edema. The wall of the left atrium is relatively thin (e.g. less than 2 to 3 mm). The pressures within the left atrium are in the range of 15 to 20 mm Hg, but in extreme conditions may elevate to 30 mm Hg or higher. During left atrial contraction approximately 5 to 7 ml of volume is displaced by the left atrial contraction. This is roughly 10 to 15% of the left atrial diastolic volume (left atrial ejection fraction).

To allow a sequential contraction of the ventricle, the ventricle will have to be paced if necessary. This function is expected to be similar to the timing cycle of a DDD pacemaker. In case a ventricular event is not sensed within a certain time, the ventricular pacemaker incorporated in the device will force a ventricular depolarization. It is expected that this technology will be incorporated into biventricular pacemakers and AICD systems. The patient will generally be required to have an intrinsic heart rate below the atrial assist device/pacemaker rate to allow effective function. This may necessitate an AV nodal ablation.

The electromagnet apparatuses of the invention are connected via insulated leads to the electromagnet control unit which includes a computer processor by which appropriate levels of current are delivered to the electromagnets to allow organ compression (and relaxation) via the magnetic induction of the electromagnets.

In addition, preferably, the embodiments of the invention monitor, for safety purposes, the temperature and pressures at the inner heart membrane, the left atrial interface. The apparatuses also allow device inactivation in case certain present parameters of left atrial press or temperature are exceeded.

Also, the apparatuses of the invention allow apparatus deactivation in case of restoration of sinus rhythm. In the event of restoration of sinus rhythm, the apparatus will function as an atrial pacing and sensing electrode.

### LIST OF REFERENCES

Here are references referred to by numerical references above.
1. Triposkiadis F, Pitsavos C, Boudoulas H, et al, Left atrial myopathy in idiopathic dilated cardiomyopathy, in Am Heart J 1994: 128,308-315 [ISI] [Medline]
2. Cryoablation of the left posterior atrial wall: 95 patients and 3 years of mean follow-up, Manasse E, Gaita F, Ghiselli S, Barbone A, Garberoglio L, Citterio E, Ornaghi D, Gallotti R, in Eur J Cardiothorac Surg, 2003, Nov. 24(5):731-40
3. Nihoyannopoulos P, Daratasakis G, Frenneaux M, et al, Diastolic function in hypertrophic cardiomyopathy: relation to exercise capacity, in J Am Coll Cardiol, 1992, 19, 536-540 [ISI][Medline]
4. Jikuhara T. Sumimoto T, Taruni N, et al, Left atrial function as a reliable predictor of exercise capacity in patients with recent myocardial infarction, in Chest 1997, 111, 922-928, [Abstract]
5. Triposkiadis F, Trikas A, Pitsavos C, et al, Relation of exercise capacity in dilated cardiomyopathy to left atrial size and systolic function, in Am J Cardiol, 1992, 30, 825-827 [ISI][Medline]
6. Changes in regional left atrial function with aging: evaluation by Doppler tissue imaging, Thomas L, Levett K, Boyd A, Leung D Y, Schiller N B, Ross D L, in Eur J Cardiothorac Surg, 2003, November, 24(5), 731-40
7. Catheter Ablation for Atrial Fibrillation in Congestive heart Failure, Volume 351:2373-2383, Dec. 2, 2004, Number 23, Li-Fern Hsu, Michel Haissaguerre, M. D. et al

The components of the atrial fibrillation treatment method and apparatus of the invention can be made from inexpensive and durable metal and plastic materials and electrical components.

As to the manner of usage and operation of the instant invention, the same is apparent from the above disclosure, and accordingly, no further discussion relative to the manner of usage and operation need be provided.

A number of target groups of patients can be assisted by employment of the atrial fibrillation treatment method and apparatus of the invention and include the following:

1. Patients with known chronic atrial fibrillation who are undergoing thoracotomy for other reasons.

2. Patients who receive an adjunct therapy during surgical radiofrequency ablation, i.e. the MAZE procedure.

3. Patients who receive an adjunct therapy during mitral valve surgery.

4. Patients who receive an adjunct therapy during coronary artery bypass grafting.

5. Possibly in all patients who are undergoing thoracotomy prophylactically for possible future use. The “leads” of the subject apparatus can be connected in the future, if needed, to a device similar t an AICD or pacemaker that allows a left atrial assist feature.

6. Patients with recurrent atrial fibrillation and congestive heart failure due to low left ventricular ejection fraction. This population group can only be treated with Amiodarone or Tikosyn (Dofetilde), and frequently these drugs are ineffective, resulting in recurrent hospitalizations and eventually death. Atrial fibrillation can decrease cardiac output by as much as 50% in patients with low ejection fraction. Ablation of atrial fibrillation shows promise in this group of patients, but due to the technical difficulties in performing this procedure, there is a need for repeat procedures, and there is an inability to restore an atrial contraction in cases of atrial myopathy. In these respects, the subject atrial fibrillation treatment method and apparatus is not likely to be widely used with this patient group. In addition, the large numbers of patients suffering from atrial fibrillation and the fact that only a few physicians are skilled in the ablation procedure will limit widespread use. Onset of atrial fibrillation is a frequent mode of deterioration and death in patients otherwise stable with biventricular pacing devices.

7. Patients with diastolic dysfunction and normal systolic function, examples being patients with left ventricular hypertrophy, hypertrophic cardiomyopathy, mitral stenosis and aortic stenosis. In this group of patients, left ventricular filling is highly dependant of the left atrial contraction. Onset of atrial fibrillation in these patients can lead to refractory congestive heart failure and inability to maintain sinus rhythm despite aggressive management.

8. Patients with rheumatic heart disease, especially mitral stenosis. Because of the associated sequelae of myocarditis, these patients develop very difficult to control atrial fibrillation and poor left atrial contraction even in sinus rhythm.

9. Some patients who have undergone pacemaker implantation and atrial fibrillation mode ablation remain symptomatic from persistent atrial fibrillation.

10. A large percentage of patients remain on coumadin for stroke prevention due to the risk from paroxysmal atrial fibrillation. Even if sinus rhythm is restored, coumadin is still required for patients with risk factors (e.g. age greater than 65 years, diabetes, hypertension, h/o CA). This is required partly due to the risk of silent atrial fibrillation and loss of left atrial contraction even in sinus rhythm due to atrial stunning and electromechanical remodeling. If left atrial contraction can be consistently restored it is possible that coumadin may be discontinued.

11. It can be used in patients with dilated cardiomyopathy and restrictive left ventricular filling pattern even if they are in sinus rhythm. Additional left atrial contractility can help improve ventricular filling.

It is apparent from the above that the present invention accomplishes all of the objects set forth by providing a new and improved atrial fibrillation treatment method and apparatus that is low in cost, relatively simple in design and operation, and which may advantageously be used to target the loss of left atrial function. With the invention, an apparatus is provided which is a simple, predictable, and effective non-pharmacological therapy that targets left atrial function. With the invention, an apparatus is provided which is simple and easy to implant and will restore contractility of the left atrium in patients who have chronic atrial fibrillation With the invention, an apparatus is provided which or sinus rhythm but poor atrial contraction due to atrial myopathy that is refractory to medical or ablative therapy. With the invention, an apparatus is provided which can be implanted epicardially at the time of cardiac surgery. With the invention, an apparatus is provided which is positioned outside the left atrium and will not itself be a source of thromboembolism, and may well result in a decrease in the risk of thromboembolism from atrial fibrillation. With the invention, an apparatus is provided which that allows programmable timing of the contraction of the left atrium to allow optimal left ventricular filling. With the invention, an apparatus is provided which allows left atrial contraction using a method of electromagnetic coupling. With the invention, an apparatus is provided which that allows incorporation into currently available biventricular pacemakers and defibrillators.

Thus, while the present invention has been shown in the drawings and fully described above with particularity and detail in connection with what is presently deemed to be the most practical and preferred embodiment(s) of the invention, it will be apparent to those of ordinary skill in the art that many modifications thereof may be made without departing from the principles and concepts set forth herein, including, but not limited to, variations in size, materials, shape, form, function and manner of operation, assembly and use.

Hence, the proper scope of the present invention should be determined only by the broadest interpretation of the appended claims so as to encompass all such modifications as well as all relationships equivalent to those illustrated in the drawings and described in the specification.

Finally, it will be appreciated that the purpose of the annexed Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. Accordingly, the Abstract is neither intended to define the invention or the application, which only is measured by the claims, nor is it intended to be limiting as to the scope of the invention in any way.

What is claimed as being new and desired to be protected by Letters Patent of the United States is as follows:

1. An apparatus for compressing and permitting relaxation of a body organ in a body, comprising:
organ compression means attached to a portion of the body organ for compressing the body organ,
compression activation means connected to said organ compression means for controlling said organ compression means for compressing the body organ and for permitting the compressed body organ to relax.

2. The apparatus of claim 1 wherein:
said organ compression means include electromagnet means, and
said compression activation means, connected to said electromagnet means, include an electromagnet control unit for controlling said electromagnet means.

3. The apparatus of claim 2 wherein said electromagnet means include:
a first electromagnet electrically connected to said electromagnet control unit, wherein said first electromagnet is placed on a first organ location on said body organ, and
a second electromagnet electrically connected to said electromagnet control unit, wherein said second electromagnet is placed on a second organ location on said body organ,
wherein said first electromagnet and said second electromagnet are independently controllable by said electromagnet control unit, such that said first electromagnet and said second electromagnet control compression of and permit relaxation of the body organ.

4. The apparatus of claim 3 wherein:
the body organ is a heart,
said first organ location is the left atrial surface of the heart,
said second organ location is the parietal pericardium of the heart, and
the first electromagnet and the second electromagnet are independently controlled by the electromagnet control unit, such that they control a left atrial contraction and permit relaxation of the left atrium.

5. The apparatus of claim 2 wherein said electromagnet means include:
an outer first electromagnet supported by a relatively stiff, nondeformable outer first-electromagnet-support member,
an inner second electromagnet supported by a flexible and deformable inner second-electromagnet-support member.

6. The apparatus of claim 5 wherein said outer first-electromagnet-support member and said inner second-electromagnet-support member are formed as a unified, integrated structure.

7. The apparatus of claim 6 wherein unified integrated structure of said outer first-electromagnet-support member and said inner second-electromagnet-support member is formed as a patch that is attached to the body organ.

8. The apparatus of claim 7, further including:
a quantity of inert gas contained inside said patch between said outer first-electromagnet-support member and said inner second-electromagnet-support member.

9. The apparatus of claim 8 wherein said patch is sutured to the left atrial surface or the left ventricular surface, such that repulsion of said second electromagnet by said first electromagnet causes an inward compression of the left atrium.

10. The apparatus of claim 2 wherein said electromagnet means include:
a flexible base member, and
a plurality of electromagnet rings attached concentrically to said flexible base member.

11. The apparatus of claim 2 wherein said electromagnet means include:
a flexible linear first electromagnet placed on a first organ location, and
a patch-like second electromagnet placed on a second organ location.

12. The apparatus of claim 11 wherein:
said flexible linear first electromagnet is placed on the epicardial surface of the left atrium, and
said patch-like second electromagnet is placed in either the coronary sinus or the intraatrial septum.

13. The apparatus of claim 1 wherein:
said organ compression means include a pneumatic bladder, and
said compression activation means, connected to said pneumatic bladder, include a pneumatic bladder control unit for controlling said pneumatic bladder.

14. The apparatus of claim 3 wherein:
the body organ is a heart,
said first organ location is the inside surface of the left atrial surface of the heart,
said second organ location is the parietal pericardium of the heart, and
the first electromagnet and the second electromagnet are independently controlled by the electromagnet control unit, such that they control a left atrial contraction and permit relaxation of the left atrium.

15. The apparatus of claim 3 wherein:
the body organ is a heart,
said first organ location is the left ventricular surface of the heart,
said second organ location is the parietal pericardium of the heart,
the first electromagnet and the second electromagnet are each comprised of a series of electromagnets independently and selectively controlled by the electromagnet control unit, such that they control a left ventricular contraction and permit relaxation of a region of the left ventricle.

16. A method for compressing and permitting relaxation of a body organ, comprising the steps of:
attaching a portion of organ compression means to the body organ,
employing compression activation means for controlling the organ compression means for compressing and permitting relaxation of the body organ.

17. The method of claim 16, comprising the steps of:
placing a first electromagnet, which is part of the organ compression means, on a first organ location of the body organ,
placing a second electromagnet, which is part of the organ compression means, on a second organ location of the body organ, and
independently controlling the first electromagnet and the second electromagnet by an electromagnet control unit, which is part of the compression activation means, such that the first electromagnet and the second electromagnet compress and permit relaxation of a portion of the body organ.

18. The method of claim 17 wherein:
the body organ is a heart,
the first organ location is the left atrial surface of the heart,
the second organ location is the parietal pericardium of the heart, and
the first electromagnet and the second electromagnet are independently controlled by the electromagnet control unit such that they control a left atrial contraction and permit relaxation of the left atrium.

19. The method of claim 17 wherein:
the body organ is a heart,
the first organ location is the left ventricular surface of the heart,
the second organ location is the parietal pericardium of the heart, and
the first electromagnet and the second electromagnet are independently controlled by the electromagnet control unit such that they control a left ventricular contraction and permit relaxation of the left ventricle.
